(19) United States
(12) Patent Application Publication
(10) Pub. No.: US 2024/0374813 A1
(43) Pub. Date: Nov. 14, 2024

(54) METHODS TO DEMONSTRATE PROGRESS TOWARDS FULL ADAPTATION AND TO PRESENT POTENTIAL IMPROVEMENTS THROUGH BEHAVIORAL CHANGES

(71) Applicant: INSULET CORPORATION, Acton, MA (US)

(72) Inventors: Joon Bok LEE, Acton, MA (US); Gregory STERN, Lexington, MA (US); Nika VARPAHOVSKY, Chestnut Hill, MA (US); Jason O’CONNOR, Acton, MA (US)

(21) Appl. No.: 18/661,020

(22) Filed: May 10, 2024

Related U.S. Application Data
(60) Provisional application No. 63/501,828, filed on May 12, 2023.

(51) Int. Cl. A61M 5/142; G16H 20/17; G16H 40/63

(52) U.S. Cl. CPC A61M 5/14248; G16H 20/17; G16H 40/63; A61M 2005/14252; A61M 2005/14292; A61M 2205/10; A61M 2205/3303; A61M 2205/3327; A61M 2205/3592; A61M 2205/502; A61M 2205/52; A61M 2230/201

(57) ABSTRACT

Exemplary embodiments calculate a user's progress towards maximum adapted system performance when utilizing a drug delivery device such as an insulin pump and display this progress to the user. Further embodiments calculate potential changes to a particular user's interaction with the system given a history of glucose and insulin deliveries and provide guidance to that user. This can be used, for example, to change how the user splits up their basal and bolus doses to improve metrics such as time-in-range, incidences of hyper- or hypo-glycemia, time in hyper- or hypo-glycemia, mean glucose, etc. It may also be used to speed the above-described adaptation process by suggesting changes that the user can make to achieve stability more quickly.

### CROSS-REFERENCE TO RELATED APPLICATIONS

This application claims priority to and the benefit of U.S. Provisional Application No. 63/501,828, filed May 12, 2023, the entirety of which is incorporated herein by reference.

### BACKGROUND

Automated drug delivery systems are used to treat a variety of conditions. Such systems include body-worn devices capable of administering therapeutics, as well as sensing and reporting physiological characteristics. Examples of such devices include (but are not limited to) devices for dispensing insulin, glucagon-like peptide (GLP-1), a combination of insulin and GLP-1, fertility drugs, white blood cell stimulating drugs, or other medicaments. These devices generally deploy a needle, conduit, cannula or other element that connects the body-worn device into the user's tissue (e.g., the subcutaneous tissue). In order determine how much of the therapeutic to dispense, the device may include a sensor (such as a glucose or ketone sensor), which may be part of the conduit or may be separate from it.

One example of an automated drug delivery system is an Automated Insulin Delivery (AID) system for people with insulin-dependence. AIDs are designed to be safe and robust for users with a wide range of varying insulin sensitivities.

One issue that arises in connection with AIDs is that, when a new user begins using the device, there may be no a priori information available regarding what a safe and effective dose of insulin might be for the user. Typically, therefore, insulin is initially biased towards underdelivery, as insulin overdelivery carries significantly greater risk rather than underdelivery (due to acute nature of hypoglycemic risk, the inability to remove insulin that is already delivered, and others). Such AID systems may increase their insulin delivery over time by adapting to each user's glucose control outcomes following this initial insulin delivery, but users may experience increased incidences of hyperglycemia until this adaptation is fully executed.

This can be an issue because users often expect an automated system to be effective “out of the box.” When a user continues to experience hyperglycemia or hypoglycemia after beginning to use such a system, they can become frustrated and might discontinue use of the system. Furthermore, a user might take certain actions that undermine the ability of the device to customize to that particular user, such as changing their diet or exercise habits. With conventional systems, there is no way for a user to determine how much these changes have affected the system's progress towards full personalization/adaptation. Accordingly, a user can become frustrated by a seeming lack of progress in achieving optimal device-based glucose control.

Moreover, even when the system has adapted and is delivering acceptable doses of insulin, further improvements may still be possible. In typical circumstances, however, methods to achieve such improvements are difficult to determine without advanced knowledge or understanding of a user's diabetes regimen. To manage their dosage, people with diabetes often rely on infrequent consultations with expert healthcare providers for guidance, which means that the user may spend a significant amount of time with sub-optimal insulin dosage patterns.

### BRIEF SUMMARY

Exemplary embodiments relate to computer-implemented methods, as well as non-transitory computer-readable mediums storing instructions for performing the methods, apparatuses configured to perform the methods, etc.

In one aspect, a method includes receiving, at a controller for an automated drug delivery device, one or more indications of drug delivery from the automated drug delivery device, calculating, using a processor of the controller, an amount of progress towards a target system performance of the automated drug delivery device based on the one or more indications of drug delivery, and displaying, on a display of the controller, the amount of progress, where the amount of progress is divided into an onboarding period, a dynamic, automatic titration period, and a target system performance period.

The titration period may refer to a period of adaptation, personalization, or customization during which certain parameters of drug delivery are determined (e.g., one or more of a user's total daily insulin amount a basal/bolus split, an amount of a basal dose, an amount of a bolus dose, timings for dosages, etc.).

The controller may be a dedicated device configured to control delivery of a drug by the drug delivery device, a separate device such as a user's personal mobile device or may be built into the drug delivery device.

The drug delivery device may attempt to adapt at predetermined intervals referred to as adaptation periods. For example, adaptation periods may occur at regular intervals (e.g., every 12 or 24 hours), or upon the occurrence of certain events (e.g., system initialization).

The target system performance period may be a period defined by a start time at which the system achieves a predetermined level of performance. The predetermined level of performance may be, for example, a level of stability in which a parameter being adapted changes by less than a predetermined threshold amount between adaptation periods. In this scenario, the drug delivery device reaches a steady state and achieves the maximum performance possible for the current adaptation scheme.

In some embodiments, the dynamic, automatic titration period may be calculated, at least in part, based on a target time-in-range for the user's glucose level over a predetermined period of time. Still further, the dynamic, automatic titration period may be calculated, at least in part, based on a comparison of a user's current mean glucose value as compared to a target mean glucose value for the user, the target mean glucose value representing an ideal diabetes control scenario.

The use of a combination of metrics (e.g., the level of stability in the parameter being adapted, time-in-range, and mean glucose value) provides a fuller and more accurate picture of the adaptation process. For example, a user might be in-range a significant amount of the time but might still have a low mean glucose value.

The onboarding period may be a predetermined fixed length of usage. The dynamic, automatic titration period may be a predetermined fixed length of usage following the onboarding period, or may be calculated based on a rate of change in a parameter being adapted (e.g., a user's total daily insulin use).

The method described above may be embodied as instructions stored on a non-transitory computer-readable storage medium storing instructions that, when executed by a controller for an automated drug delivery device, cause the controller to perform the above method. The automated drug delivery device may include a reservoir storing a liquid drug, a delivery system for delivering the liquid drug to a user, a processor for calculating dosage amounts for the liquid drug, and a transmitter/receiver configured to transmit the dosage amounts. The controller may include a transmitter/receiver, a display, a processor, and a memory storing instructions for performing the above-described method.

In another embodiment, which may be used separately or in conjunction with any of the embodiments described above, a method includes receiving, at a controller for an automated drug delivery device, one or more blood glucose readings from a sensor of the automated drug delivery device, determining, based on the one or more blood glucose readings, that a sub-optimal glucose control condition exists, and displaying, on a display of the controller, a recommendation based on the sub-optimal glucose control condition.

Determining that a sub-optimal glucose control condition exists may include one or more of: determining that an amount of time that a user has spent in a hyperglycemic range exceeds a predetermined hyperglycemic threshold amount, determining that an amount of time that a user has spent in a hypoglycemic range exceeds a predetermined hypoglycemic threshold amount, determining that the user has experienced more than a predetermined number of instances of glucose concentrations above a predetermined upper threshold amount, determining that the user has experienced more than a predetermined number of instances of glucose concentrations below a predetermined lower threshold amount, and/or determining that a sub-optimal glucose control condition exists includes determining that the user has experienced an average glucose concentration that is higher than a predetermined average concentration threshold amount.

The recommendation may include one or more of an expected best-case blood glucose control scenario, a suggested change to the user's basal dose, or a suggested change to the user's bolus dose.

Alternatively or in addition to the above recommendations, the method may involve displaying a relationship between the user's average bolus per day and/or bolus ratio to the user's time-in-range.

In further embodiments, a non-transitory computer-readable storage medium may be provided, the computer-readable storage medium including instructions that when executed by a controller for an automated drug delivery device, cause the controller to perform the above-described method.

In still further embodiments, the method may be performed in a system that includes an automated drug delivery device and a controller. The automated drug delivery device may include a sensor configured to detect a blood glucose level of a user, a processor for calculating the user's blood glucose level, and a transmitter/receiver configured to transmit the dosage amounts. The controller may include a transmitter/receiver, a display, a processor, and a memory storing instructions that, when executed by the processor, configure the controller to perform the above-described method.

Other technical features may be readily apparent to one skilled in the art from the following figures, descriptions, and claims.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 depicts an exemplary automated drug delivery system in accordance with one embodiment.

FIG. 2A depicts an interface displaying a user's progress towards a target system performance in accordance with one embodiment.

FIG. 2B depicts an interface displaying a user's progress towards a target system performance in accordance with one embodiment.

FIG. 2C depicts an interface displaying a user's progress towards a target system performance in accordance with one embodiment.

FIG. 3 is a flowchart depicting exemplary logic for performing a computer-implemented method for displaying a progress towards a target system performance according to an exemplary embodiment.

FIG. 4 is a table showing how different drug delivery recommendations may be calculated in accordance with one embodiment.

FIG. 5 is a flowchart depicting exemplary logic for performing a computer-implemented method for making recommendations regarding drug delivery according to an exemplary embodiment.

FIG. 6 depicts an exemplary insights interface displaying a user's insulin basal/bolus split in accordance with one embodiment.

FIG. 7 depicts an exemplary insights interface displaying how the user's bolus ratio affects the user's time-in-range in accordance with one embodiment.

FIG. 8 depicts an exemplary insights interface displaying an average number of boluses per day in accordance with one embodiment.

FIG. 9 depicts an illustrative computer system architecture that may be used to practice exemplary embodiments described herein.

### DETAILED DESCRIPTION

Exemplary embodiments calculate a user's progress towards maximum adapted system performance when utilizing a drug delivery device such as an insulin pump and display this progress to the user.

Conventional systems continuously adapt to changes in the user's behavior and/or blood glucose level, effectively having an infinite adaptation period. Exemplary embodiments delineate the user experience into an initial onboarding period during which the drug is expected to be delivered sub-optimally (e.g., underdelivered, in the case of insulin), an adaptation period during which performance improves, and a target performance period during which the device is effectively adapted to the user's current lifestyle. By providing the user with an indication of their progress through each stage, the user is kept better informed of the performance that they can expect from the device.

After the system is adapted to a particular user, this does not necessarily indicate that the user will no longer experience conditions such as hyper/hypoglycemia Rather, full adaptation refers to when the drug delivery device achieves a predetermined level of stability, or approaches within a predetermined threshold of optimal diabetes control (e.g., achieving a certain amount of time in a target blood glucose range, referred to as “time-in-range”, or target blood glucose levels such as mean glucose or estimated average glucose). Further improvements in the user's diabetes management may be possible but may require changes to the user's lifestyle (e.g., their diet and/or exercise regimen).

Thus, further embodiments, which may be used separately or in conjunction with the above-summarized adaptation embodiments, calculate potential changes to a particular user's interaction with the system given a history of glucose and insulin deliveries, and provide guidance to that user. This can be used, for example, to change how the user splits up their basal and bolus doses to improve metrics such as time-in-range, incidences of hyper- or hypo-glycemia, time in hyper- or hypo-glycemia, mean glucose, etc. It may also be used to speed the above-described adaptation process by suggesting changes that the user can make to achieve stability more quickly.

### A Note on Data Privacy

Some embodiments described herein make use of training data or metrics that may include information voluntarily provided by one or more users. In such embodiments, data privacy may be protected in a number of ways.

For example, the user may be required to opt in to any data collection before user data is collected or used. The user may also be provided with the opportunity to opt out of any data collection. Before opting in to data collection, the user may be provided with a description of the ways in which the data will be used, how long the data will be retained, and the safeguards that are in place to protect the data from disclosure.

Any information identifying the user from which the data was collected may be purged or disassociated from the data. In the event that any identifying information needs to be retained (e.g., to meet regulatory requirements), the user may be informed of the collection of the identifying information, the uses that will be made of the identifying information, and the amount of time that the identifying information will be retained. Information specifically identifying the user may be removed and may be replaced with, for example, a generic identification number or other non-specific form of identification.

Once collected, the data may be stored in a secure data storage location that includes safeguards to prevent unauthorized access to the data. The data may be stored in an encrypted format. Identifying information and/or non-identifying information may be purged from the data storage after a predetermined period of time.

Although particular privacy protection techniques are described herein for purposes of illustration, one of ordinary skill in the art will recognize that privacy protected in other manners as well. Further details regarding data privacy are discussed below in the section describing network embodiments.

Assuming a user's privacy conditions are met, exemplary embodiments may be deployed in a wide variety of messaging systems, including messaging in a social network or on a mobile device (e.g., through a messaging client application or via short message service), among other possibilities. An overview of exemplary logic and processes for engaging in synchronous video conversation in a messaging system is next provided.

### Exemplary Embodiments

As an aid to understanding, a series of examples will first be presented before detailed descriptions of the underlying implementations are described. It is noted that these examples are intended to be illustrative only and that the present invention is not limited to the embodiments shown.

Reference is now made to the drawings, wherein like reference numerals are used to refer to like elements throughout. In the following description, for purposes of explanation, numerous specific details are set forth in order to provide a thorough understanding thereof. However, the novel embodiments can be practiced without these specific details. In other instances, well known structures and devices are shown in block diagram form in order to facilitate a description thereof. The intention is to cover all modifications, equivalents, and alternatives consistent with the claimed subject matter.

In the Figures and the accompanying description, the designations “a” and “b” and “c” (and similar designators) are intended to be variables representing any positive integer. Thus, for example, if an implementation sets a value for a=5, then a complete set of components 122 illustrated as components 122-1 through 122-a may include components 122-1, 122-2, 122-3, 122-4, and 122-5. The embodiments are not limited in this context.

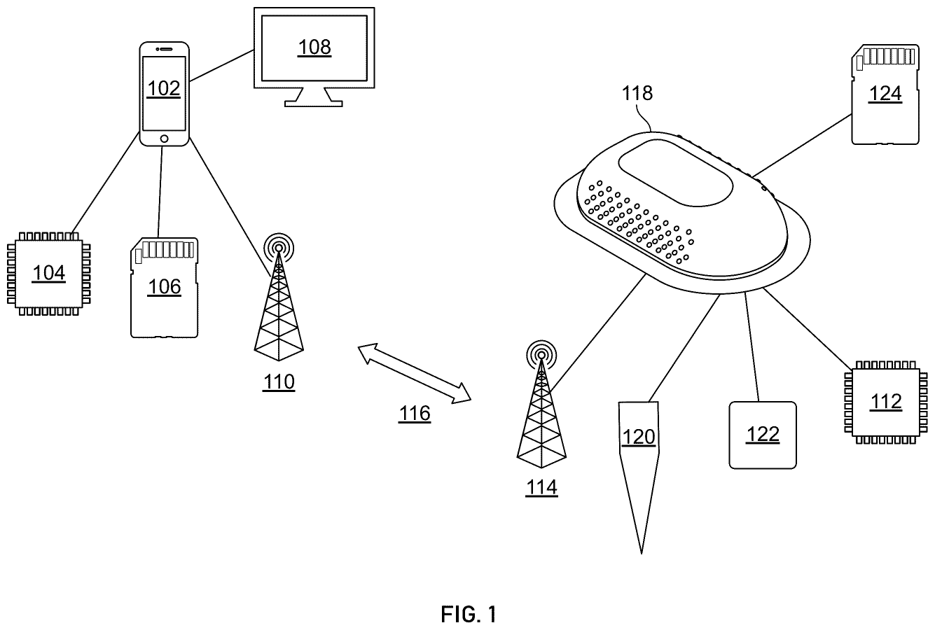

As shown in FIG. 1, exemplary embodiments may be performed in a drug delivery system that includes a controller/smartphone 102 and an automated insulin delivery device 118. Although FIG. 1 is described in connection with a particular type of drug delivery device (the automated insulin delivery device 118), embodiments are not so limited. Instead, it is contemplated that exemplary embodiments may be used with any drug delivery device suitable for dispensing a drug (such as a liquid drug) for which dosages may be adapted to a particular user.

The controller/smartphone 102 may be a dedicated hardware device specifically configured for use with the automated insulin delivery device 118. Alternatively, the controller/smartphone 102 may be a general purpose device, such as a user's personal computer or mobile device. Although FIG. 1 depicts the controller/smartphone 102 as a separate device from the automated insulin delivery device 118, in exemplary embodiments some or all of the functionality of the controller/smartphone 102 may be combined with the automated insulin delivery device 118 (e.g., the controller/smartphone 102 and automated insulin delivery device 118 may share processing and/or data storage capabilities, or the display 108 may be provided on the automated insulin delivery device 118).

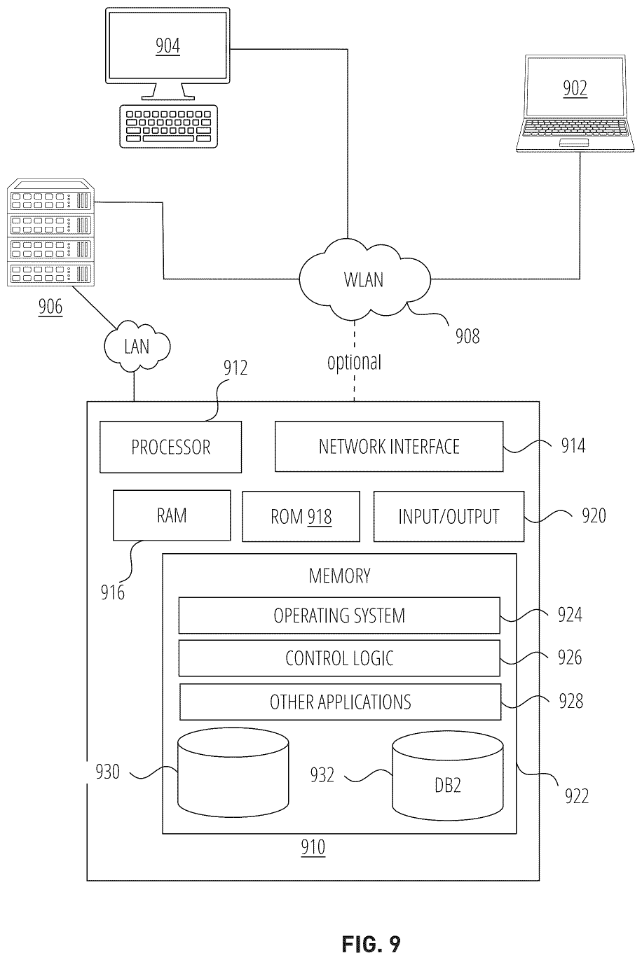

The controller/smartphone 102 may include a processor 104, a storage medium 106, a display 108, and/or a transmitter/receiver 110. Suitable types of hardware are discussed in more detail below in connection with FIG. 9.

The automated insulin delivery device 118 may include a processor 112, a transmitter/receiver 114, a storage medium 124, a delivery system 120, and a sensor 122. The delivery system 120 may include a reservoir configured to store a liquid drug and a needle or cannula configured to be deployed into a user (e.g., subcutaneously) to deliver the liquid drug from the reservoir. The delivery system 120 may further include a pump for moving the drug from the reservoir into the user.

The sensor 122 may be any suitable type of sensor for detecting a condition of the user; for example, an insulin delivery device might utilize a continuous glucose monitor (CGM) or ketone sensor.

Information from the sensor 122 (such as glucose readings) and/or the delivery system 120 (such as dosage amounts and times) may be transmitted between the transmitter/receiver 110 and the transmitter/receiver 114 via a link 116. The link 116 may be any suitable type of link, such as a wireless link (e.g., via a short-range or long-range transmission protocol, WiFi, etc.).

In typical embodiments, an AID system that is designed for a wide range of potential users will have an “onboarding” period where it behaves very conservatively, followed by a longer period where it will adapt to the user's insulin needs over time. Thus, the full journey of adaptivity journey may be defined into different phases, such as:
1. Onboarding
2. Ongoing adaptation
3. Maximum or target or optimized or steady-state performance reached

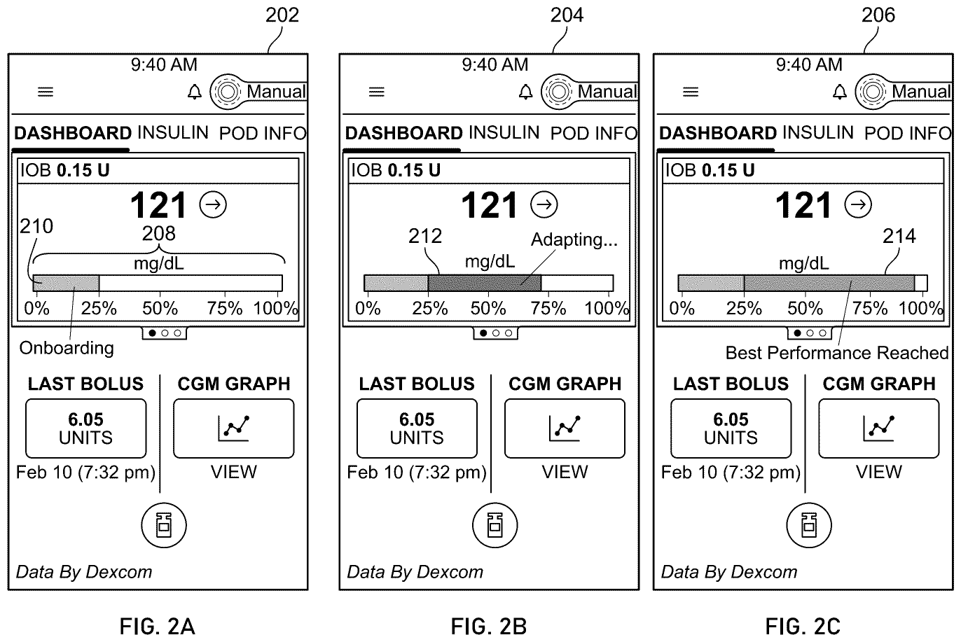

Exemplary embodiments show the user's progress through these phases on a display of the controller, in order to give a user a better sense of the performance that they should expect from the drug delivery device. FIG. 2A-FIG. 2C depict graphical user interfaces including one example of a technique for displaying this progress.

In some embodiments, the onboarding period may be a fixed period that is defined by design constraints and user safety needs. However, the rate of ongoing adaptation and progress to maximum/target performance reached through adaptation may vary depending on the system behavior and user interactions with the system.

This progress over the period of ongoing adaptation, as well an indication of whether maximum performance is reached, may be calculated by a variety of factors.

In one embodiment, this may be simply calculated as a fixed duration following onboarding. Specifically, design of the adaptivity concept for an AID system may define parameters on how much the user must utilize the system before the system reaches maximum/target performance. This may be calculated as shown in Equation 1 below

$$P = ( 1 - \frac{{T}_{ob} - D}{{T}_{ob}} ) \cdot 100 \qquad Equation 1$$

Here, P represents the progress from onboarding period to maximum/target performance in % of maximum performance, D represents the duration of system use, and T_{ob} represents the period of system use when onboarding period has completed. an exemplary value of T_{ob} may be 48 hours, representing 48 hours of system use that is required before the adaptivity scheme in AID system allows maximum performance.

In another proposed embodiment, this progress may be calculated by the rate of change in the parameter for the AID system that is being adapted. For instance, if the user's TDI, or total daily insulin, is the parameter that is being adapted, the amount of this parameter that is being changed through this ongoing adaptation may be converted to displayed progress towards maximum/target adaptivity as follows:

$$P = ( 1 - \frac{{TDI}_{n} - {TDI}_{n - 1}}{{TDI}_{n}} ) \cdot 100 \qquad Equation 2$$

Here, TDIₙ is the TDI value for the n^{th} iteration of adaptivity (or adaptivity period), and the smaller the change between the different periods of adaptivity, the closer the system has come to reaching “steady state,” or the maximum performance possible for the current adaptivity scheme.

In some embodiments, the system may also combine the change in TDI value as well as the difference in the user's current mean glucose, versus the best mean glucose concentrations that would be expected for the user when using an automated drug delivery system. This may be represented as follows:

$$P = ( 1 - \frac{{TDI}_{n} - {TDI}_{n - 1}}{{TDI}_{n}} - min ( \frac{C \overline{G} {M}_{n} - CG {\overline{M}}_{max}}{C \overline{G} {M}_{1} - CG {\overline{M}}_{max}} , 0 ) ) \cdot 100 \qquad Equation 3$$

In this equation,
CG̅Mₙ is the mean glucose for the n^{th} iteration of the system,
CGM̅ₘₐₓ represents the best case mean glucose control performance expected when utilizing the AID system, and
CG̅M₁ represents the mean glucose concentration after the first use of the system.

CGM̅ₘₐₓ may represent the theoretical best performance that a drug delivery system can achieve. For example, for an AID system the CGM̅ₘₐₓ may be based on an optimal HbA1c (e.g., 6.5%).

CG̅M₁ may represent the mean glucose from the first use of the AID system (e.g., the first period of adaptivity). For example, adaptation may not begin until at least 48 hours of data has been collected, and CG̅M₁ may represent the mean glucose over that initial 48-hour period.

CG̅M₁ is likely to represent a worst-case scenario. In some cases, the first cycle may be an abnormality. Accordingly, in some embodiments the value of CG̅M₁ may take on the worst mean glucose value observed for any period of adaptivity.

Once this progress to adaptivity is calculated, this may be displayed to the user through a variety of visual cues. In one example, this may be represented to the user visually in the user interface of the AID system (e.g., on the display 108 of the controller/smartphone 102) as shown in FIG. 2A-FIG. 2B.

In FIG. 2A, an onboarding screen 202 is presented showing a progress bar 208. In this case, the user is still in the onboarding process. Accordingly, the progress bar 208 is only partially filled. The portion of the progress bar 208 that is filled corresponds to an onboarding portion 210. In this example, onboarding is estimated to take 25% of the total amount of time before the AID is adapted to the user (this may be a predetermined estimate). The onboarding portion 210 may fill the progress bar 208 up to this predetermined estimate (e.g., 25%). The onboarding portion may proceed from 0% to 25% (in this example) based on the progress indicated by Equation 1 above. While onboarding, the onboarding portion 210 may be visually distinguished from the unfilled portion of the progress bar 208 and any other portions of the progress bar 208 (such as the titrating portion 212).

In FIG. 2B, onboarding has completed and a titrating screen 204 is shown. In this case, the progress bar 208 is filled up to 25% by the progress bar 208, and another titrating portion 212 is shown. The titrating portion 212 may fill the remainder of the bar based on progress through the adaptation period as described (for example) in Equations 2 and 3. While titrating, the titrating portion 212 may be visually distinguished from the unfilled portion of the progress bar 208 (e.g., the right-hand section) and any other portions of the progress bar 208 (such as the onboarding portion 210).

For both the onboarding portion 210 and the titrating portion 212, the length of the bar for the adapting period may be represented by the parameter P above.

When the system has adapted to a particular user, the titrating portion 212 may change in appearance (e.g., by changing color) to a target performance portion 214. The target performance portion 214 is shown in the target performance screen 206 of FIG. 2C. In some embodiments, the target performance portion 214 may fill the progress bar 208 to 100%. In others, the target performance portion 214 may fill the progress bar 208 to a predetermined amount that is less than 100% in order to visually indicate that, although target or steady-state performance has been reached, the drug delivery device will continue adapting to changes in the user's lifestyle and that further improvement may be had by altering the user's diet and/or exercise regimen. In such case, a target or steady-state may rely on the assumption that the user's lifestyle will remain consistent over time (e.g., the user does not drastically change their diet or exercise patterns).

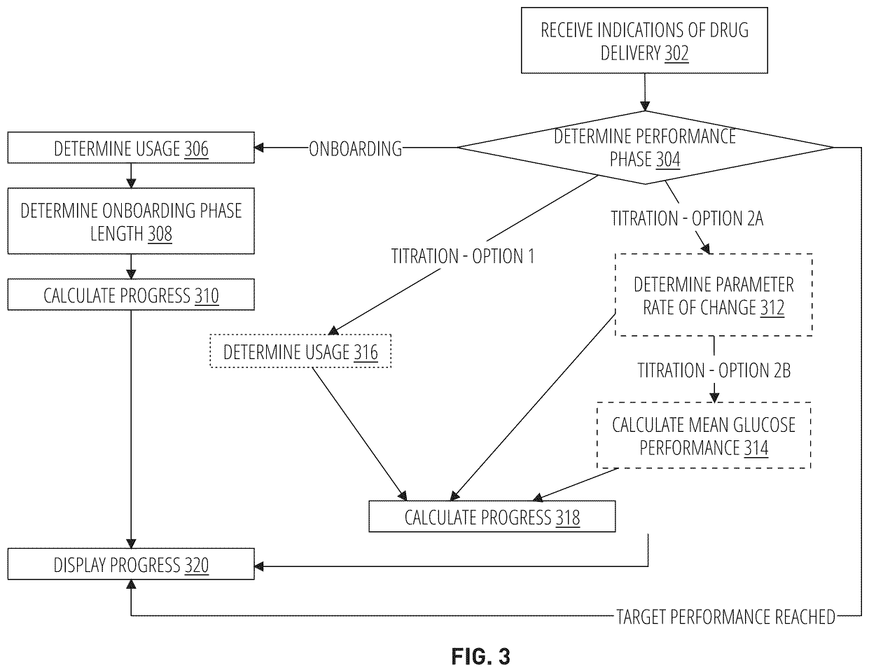

FIG. 3 is a flowchart depicting exemplary logic for performing a computer-implemented method according to an exemplary embodiment. The logic may be embodied as instructions stored on a computer-readable medium configured to be executed by a processor. The logic may be implemented by a suitable computing system configured to perform the actions described below.

Although the example routine depicts a particular sequence of operations, the sequence may be altered without departing from the scope of the present disclosure. For example, some of the operations depicted may be performed in parallel or in a different sequence that does not materially affect the function of the routine. In other examples, different components of an example device or system that implements the routine may perform functions at substantially the same time or in a specific sequence.

In some embodiments, the method includes receiving indications of drug delivery at block 302. The indications of drug delivery may be received at the controller from the drug delivery device. The drug delivery device may record each dose delivered by the delivery system in its memory, including the time and amount of drug dispensed from the reservoir. This information may be transmitted by the drug delivery device's transmitter/receiver over the link, and may be received at the controller's transmitter receiver and loaded into the controller's memory. The indications of drug delivery may be transmitted as the drug is dispensed, or may be batched and transmitted at predetermined or dynamically determined intervals.

In some embodiments, the method includes determining a current performance phase at decision block 304. Initially, the drug delivery device may be in the onboarding phase and may remain there until the drug delivery device has experienced a certain amount of usage, as indicated by Equation 1 above. Thus, if it is determined that the drug delivery device is still in the onboarding phase at decision block 304, then processing may proceed to block 306 where the current amount of usage of the drug delivery device is determined. For example, the amount of time since the drug delivery device was initialized may be determined, or an amount of time that the drug delivery device was in an active state may be added up.

The method further includes determining onboarding phase length at block 308. The onboarding phase length may be a predetermined length of time that may be stored in the memory of the drug delivery device and/or the controller. At block 310, progress towards the end of the onboarding phase may be calculated according to Equation 1. Then, at block 316 this progress may be shown on a graphical user interface, such as by updating a progress bar as shown in FIG. 2A.

If, at decision block 304, it is determined that the current phase is the titration phase (i.e., the drug delivery device has completed the onboarding phase but has not yet reached a target performance threshold), then the next processing step may depend on whether the titration phase is configured to extend for a predetermined length of time (Option 1), whether the titration phase continues until a parameter being adjusted reaches a steady state (Option 2A), or whether the parameter steady state is used in conjunction with other metrics such as mean glucose performance or time-in-range (Option 2B).

In the case of option 1, processing may proceed to block 316 where the current usage of the drug delivery device may be determined. Usage may be determined in a similar manner to block 306. Processing then proceeds to block 318 and progress towards target performance may be calculated (e.g., in a manner similar to that described in connection with Equation 1, with the predetermined length of the titration phase being substituted for the predetermined length of the onboarding phase).

In the case of option 2A, processing may proceed to block 312 where the parameter rate of change may be calculated and progress may be determined (block 318) according to Equation 2 above.

In the case of option 2B, processing also proceeds to block 312 and the parameter rate of change is determined, but this information is combined at block 314 with other information such as mean blood glucose performance. Processing then proceeds to block 318, where progress is determined according to Equation 3 (or a variation if information other than mean blood glucose is used).

Processing then proceeds to block 320, where the user interface is updated as shown in FIG. 2B.

If, at decision block 304, it is determined that the system has reached its target performance level, then processing may proceed directly to block 320 and the user interface may be updated as shown in FIG. 2C.

According to further embodiments, suboptimal glucose control conditions may be detected during the onboarding/titration processes described above, and/or after the target performance level is reached. The system may offer recommendations for improving the system's adaptation time or for improving the user's disease management.

In particular, insulin pump systems that are used in conjunction with continuous glucose monitor (CGM) sensors, such as automated insulin delivery systems, maintain a record of key elements of each user's insulin therapy, including the following:
Automated/manual basal insulin delivery history
Automated/manual bolus insulin delivery history
Glucose readings

An insulin pump system may be able to determine that the user may be experiencing sub optimal glucose control outcomes, and recommend changes in the user's behavior to address such cases. The process of calculating such recommendations may occur in two components.

### Part 1: Detection of Sub Optimal Glucose Control

Several conditions may result in sub optimal glucose control. The insulin pump system may test for such conditions every certain period, such as 24 hours, or every requisite time where the pump insertion site must be replaced, and detect that the user has experienced sub optimal glucose control if one, some, or all conditions are met. These conditions may include:
Time in Hyperglycemia: The user's time in hyperglycemic range (>180 mg/dL) may be greater than a threshold (such as 30%) since last assessment
Time in hypoglycemia: The user's time in hypoglycemic range (<70 mg/dL) may be greater than a threshold (such as 4%) since last assessment
Individual hyper- or hypoglycemic incidence: The user has experienced a certain number, such as more than one, of instances of glucose concentrations above or below a certain threshold (such as 300 mg/dL and/or 40 mg/dL) since last assessment
High mean glucose: The user has experienced an average glucose concentration that is higher than a certain threshold (such as 160 mg/dL) since last assessment

### Part 2: Recommendation for Changes in User Behavior

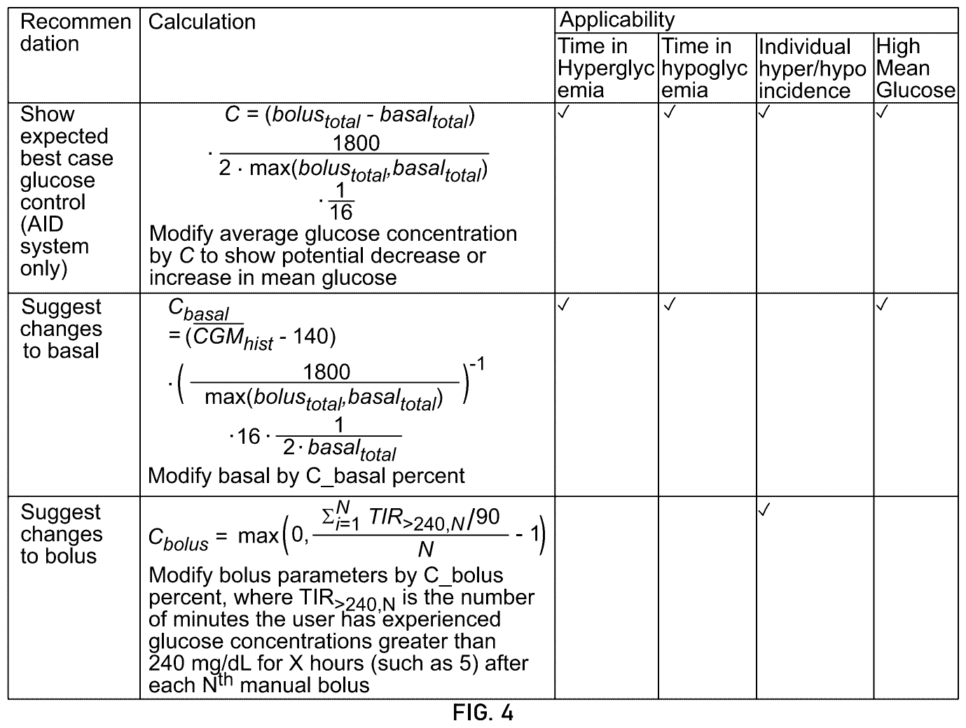

Once the insulin pump system detects one or more of preceding conditions for sub optimal control, the system may provide recommendations for changes in user behavior based on the conditions that are met. These recommendations may vary based on the condition that is met. Some example recommendations are shown in FIG. 4.

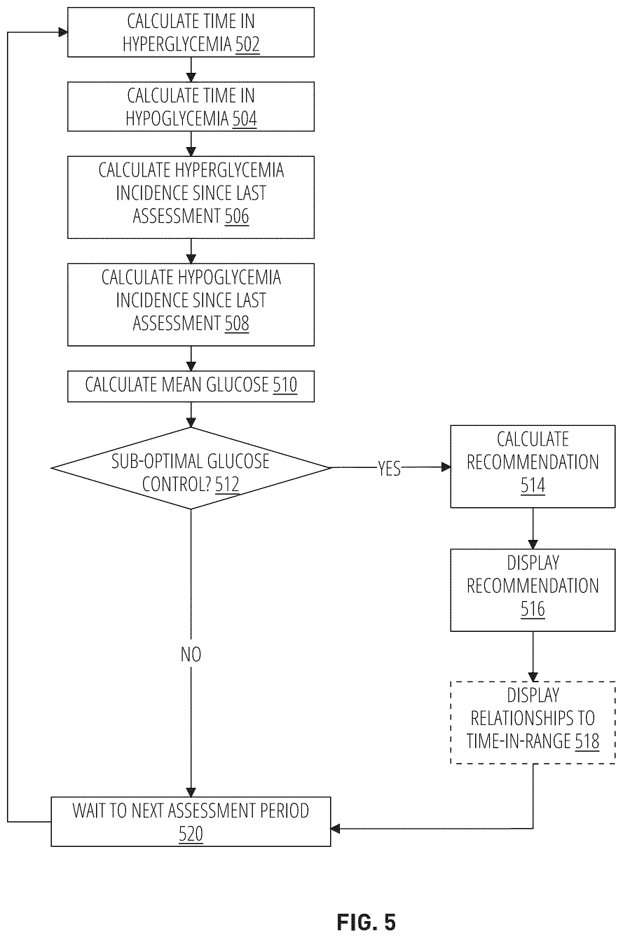

FIG. 5 is a flowchart depicting exemplary logic for performing a computer-implemented method for making recommendations to address sub-optimal glucose control according to an exemplary embodiment. The logic may be embodied as instructions stored on a computer-readable medium configured to be executed by a processor. The logic may be implemented by a suitable computing system configured to perform the actions described below.

Although the example routine depicts a particular sequence of operations, the sequence may be altered without departing from the scope of the present disclosure. For example, some of the operations depicted may be performed in parallel or in a different sequence that does not materially affect the function of the routine. In other examples, different components of an example device or system that implements the routine may perform functions at substantially the same time or in a specific sequence.

According to some examples, the method includes calculating time in hyperglycemia at block 502. Time in hyperglycemia may be determined by accessing measurements from the sensor of the drug delivery device (which may be stored in the memory of the drug delivery device and/or the controller) and accumulating the amount of time that the user's blood glucose concentration was above a predetermined hyperglycemic threshold (e.g., >180 mg/dL)

According to some examples, the method includes calculating time in hypoglycemia at block 504. Time in hypoglycemia may be determined by accessing measurements from the sensor of the drug delivery device and accumulating the amount of time that the user's blood glucose concentration was below a predetermined hypoglycemic threshold (e.g., <70 mg/dL)

According to some examples, the method includes calculating hyperglycemia incidence since last assessment at block 506, and hypoglycemia incidence since last assessment at block 508. These incidences may represent the number of times that the user's glucose concentrations fell above or below a predetermined hyper- or hypo-glycemia threshold, respectively. The thresholds for hyper- or hypo-glycemia incidences may be different from those used in blocks 502 and 504. For example, a hyperglycemia instance threshold for purposes of block 506 might be 300 mg/dL, whereas a hypoglycemia instance threshold for purposes of block 508 might be 40 mg/dL.

According to some examples, the method includes calculating mean glucose at block 510. Mean glucose may be an average glucose concentration over a period of time (e.g., between assessment periods) as determined by the drug delivery device's sensor.

According to some examples, the method includes determining whether any of the conditions in blocks 502-510 indicate sub-optimal glucose control at decision block 512.

For example, the amount of time in the hyperglycemic range, as determined at block 502, may be expressed as a percentage of total time that the device was active or a percentage of total sensor measurements. If the percentage is greater than a predetermined hyperglycemic threshold (e.g., 30%), this may be flagged as a sub-optimal glucose control condition.

Similarly, if the time in hypoglycemic range is greater than a predetermined hypoglycemic threshold (e.g., >4%), if the hyper- or hypo-glycemic incidences exceed a predetermined threshold number (e.g., >1), or if the user's average glucose concentration exceeds a predetermined threshold amount (e.g., 160 mg/dL) since the last assessment, these may also be flagged as sub-optimal glucose control conditions.

If it is determined at decision block 512 that no sub-optimal glucose control condition exists, then processing may proceed to block 520 and the system may wait a predetermined period of time corresponding to the assessment period (e.g., 12, 24, or 48 hours).

If, on the other hand, it is determined at decision block 512 that a sub-optimal glucose control condition exists, then a recommendation may be calculated at block 514. The recommendation may be dependent on the type of sub-optimal glucose control condition detected in decision block 512, as indicated by the chart in FIG. 4.

For example, the system may determine how the user's mean glucose would change if the user's basal/bolus split were adjusted towards 50% using the equation in the first row of FIG. 4. This may encourage the user to adjust their bolus behavior on their own. Alternatively or in addition, the system may recommend a change to the user's next basal or bolus dose based on the equations in rows 2 and of the table of FIG. 4, respectively.

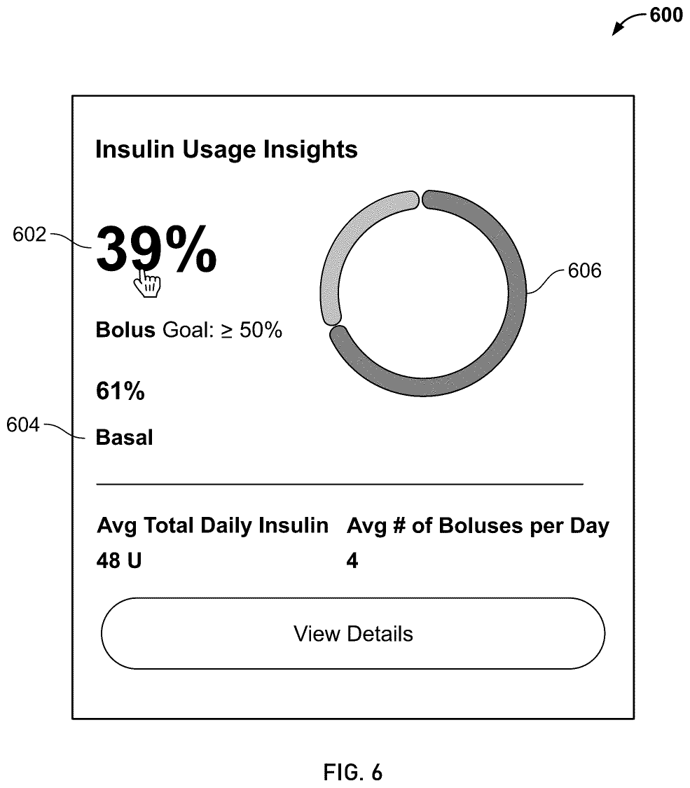

According to some examples, the method includes displaying the recommendation at block 516. The recommendation may be displayed on a display of the controller. The recommendation may be displayed in a graphical user interface, as shown for example in FIG. 6-FIG. 8.

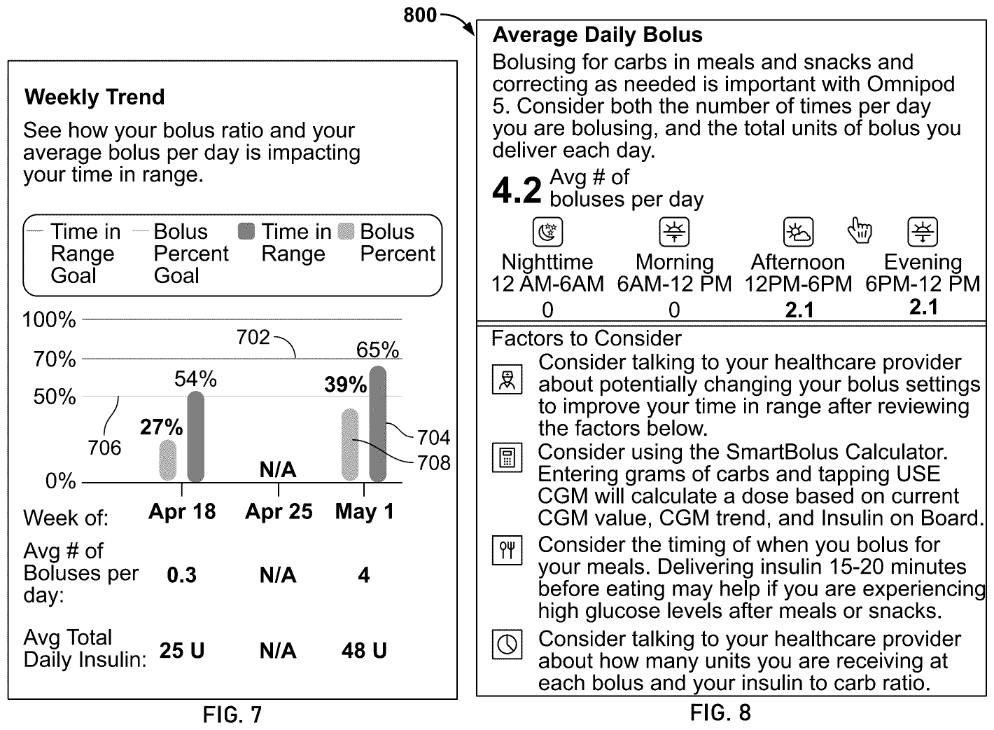

Optionally, the method may include displaying relationships between the user's basal/bolus dosing behavior and time-in-range at block 518. For example, the system may display a chart, such as the one shown in FIG. 7, that tracks the proportion of the user's total daily insulin (TDI) delivered as a bolus dose, as compared to an ideal 50/50 basal/bolus split. The chart may indicate how the user's basal/bolus split has changed over time, and how this affects the user's amount of time-in-range for glucose control.

The system may then wait for the next assessment period at block 520 and repeat the above-described process.

FIG. 6-FIG. 8 display exemplary interfaces suitable for making recommendations or offering insights into insulin usage. FIG. 6 depicts an exemplary insights GUI 600 that shows the user's basal/bolus split over the last assessment period. The insights GUI 600 displays the bolus amount 602 and the basal amount 604, and indicates that an ideal split would include a bolus amount 602 at a level of at least 50%. A ratio display 606 graphically depicts the ratio between the bolus amount 602 and basal amount 604.

FIG. 7 depicts an example of a GUI for connecting the basal/bolus ratio to the amount of time-in-range for the user. A line representing a time in range goal 702 graphically displays a target amount of time-in-range that a user wishes to (or should) achieve. Different bars for time in range 704 show how closely the user has approached the time in range goal 702 over each assessment period. A bolus percent goal 706 shows what percentage of TDI would be delivered in bolus doses in an ideal split, while bars representing the user's bolus percent 708 show how closely the user has approximated this ideal split over each assessment period. With this display, the user can see how changing their bolus percent 708 moves them closer or further away from their time in range goal 702.

FIG. 8 depicts an average daily bolus interface 800. The average daily bolus interface 800 includes information such as the user's average number of bolus doses per day, the timing of the bolus doses, and relevant information to help the user better determine how and when to administer a bolus dose.

FIG. 9 illustrates one example of a system architecture and data processing device that may be used to implement one or more illustrative aspects described herein in a standalone and/or networked environment. Various network nodes, such as the data server 910, web server 906, computer 904, and laptop 902 may be interconnected via a wide area network 908 (WAN), such as the internet. Other networks may also or alternatively be used, including private intranets, corporate networks, LANs, metropolitan area networks (MANs) wireless networks, personal networks (PANs), and the like. Network 908 is for illustration purposes and may be replaced with fewer or additional computer networks. A local area network (LAN) may have one or more of any known LAN topology and may use one or more of a variety of different protocols, such as ethernet. Devices data server 910, web server 906, computer 904, laptop 902 and other devices (not shown) may be connected to one or more of the networks via twisted pair wires, coaxial cable, fiber optics, radio waves or other communication media.

Computer software, hardware, and networks may be utilized in a variety of different system environments, including standalone, networked, remote-access (aka, remote desktop), virtualized, and/or cloud-based environments, among others.

The term “network” as used herein and depicted in the drawings refers not only to systems in which remote storage devices are coupled together via one or more communication paths, but also to stand-alone devices that may be coupled, from time to time, to such systems that have storage capability. Consequently, the term “network” includes not only a “physical network” but also a “content network,” which is comprised of the data—attributable to a single entity—which resides across all physical networks.

The components may include data server 910, web server 906, and client computer 904, laptop 902. Data server 910 provides overall access, control and administration of databases and control software for performing one or more illustrative aspects described herein. Data server 910 may be connected to web server 906 through which users interact with and obtain data as requested. Alternatively, data server 910 may act as a web server itself and be directly connected to the internet. Data server 910 may be connected to web server 906 through the network 908 (e.g., the internet), via direct or indirect connection, or via some other network. Users may interact with the data server 910 using remote computer 904, laptop 902, e.g., using a web browser to connect to the data server 910 via one or more externally exposed web sites hosted by web server 906. Client computer 904, laptop 902 may be used in concert with data server 910 to access data stored therein, or may be used for other purposes. For example, from client computer 904, a user may access web server 906 using an internet browser, as is known in the art, or by executing a software application that communicates with web server 906 and/or data server 910 over a computer network (such as the internet).

Servers and applications may be combined on the same physical machines, and retain separate virtual or logical addresses, or may reside on separate physical machines. FIG. 9 illustrates just one example of a network architecture that may be used, and those of skill in the art will appreciate that the specific network architecture and data processing devices used may vary, and are secondary to the functionality that they provide, as further described herein. For example, services provided by web server 906 and data server 910 may be combined on a single server.

Each component data server 910, web server 906, computer 904, laptop 902 may be any type of known computer, server, or data processing device. Data server 910, e.g., may include a processor 912 controlling overall operation of the data server 910. Data server 910 may further include RAM 916, ROM 918, network interface 914, input/output interfaces 920 (e.g., keyboard, mouse, display, printer, etc.), and memory 922. Input/output interfaces 920 may include a variety of interface units and drives for reading, writing, displaying, and/or printing data or files. Memory 922 may further store operating system software 924 for controlling overall operation of the data server 910, control logic 926 for instructing data server 910 to perform aspects described herein, and other application software 928 providing secondary, support, and/or other functionality which may or may not be used in conjunction with aspects described herein. The control logic may also be referred to herein as the data server software control logic 926. Functionality of the data server software may refer to operations or decisions made automatically based on rules coded into the control logic, made manually by a user providing input into the system, and/or a combination of automatic processing based on user input (e.g., queries, data updates, etc.).

Memory 1122 may also store data used in performance of one or more aspects described herein, including a first database 932 and a second database 930. In some embodiments, the first database may include the second database (e.g., as a separate table, report, etc.). That is, the information can be stored in a single database, or separated into different logical, virtual, or physical databases, depending on system design. Web server 906, computer 904, laptop 902 may have similar or different architecture as described with respect to data server 910. Those of skill in the art will appreciate that the functionality of data server 910 (or web server 906, computer 904, laptop 902) as described herein may be spread across multiple data processing devices, for example, to distribute processing load across multiple computers, to segregate transactions based on geographic location, user access level, quality of service (QOS), etc.

One or more aspects may be embodied in computer-usable or readable data and/or computer-executable instructions, such as in one or more program modules, executed by one or more computers or other devices as described herein. Generally, program modules include routines, programs, objects, components, data structures, etc. that perform particular tasks or implement particular abstract data types when executed by a processor in a computer or other device. The modules may be written in a source code programming language that is subsequently compiled for execution, or may be written in a scripting language such as (but not limited to) HTML or XML. The computer executable instructions may be stored on a computer readable medium such as a nonvolatile storage device. Any suitable computer readable storage media may be utilized, including hard disks, CD-ROMs, optical storage devices, magnetic storage devices, and/or any combination thereof. In addition, various transmission (non-storage) media representing data or events as described herein may be transferred between a source and a destination in the form of electromagnetic waves traveling through signal-conducting media such as metal wires, optical fibers, and/or wireless transmission media (e.g., air and/or space). various aspects described herein may be embodied as a method, a data processing system, or a computer program product. Therefore, various functionalities may be embodied in whole or in part in software, firmware and/or hardware or hardware equivalents such as integrated circuits, field programmable gate arrays (FPGA), and the like. Particular data structures may be used to more effectively implement one or more aspects described herein, and such data structures are contemplated within the scope of computer executable instructions and computer-usable data described herein.

The components and features of the devices described above may be implemented using any combination of discrete circuitry, application specific integrated circuits (ASICs), logic gates and/or single chip architectures. Further, the features of the devices may be implemented using microcontrollers, programmable logic arrays and/or microprocessors or any combination of the foregoing where suitably appropriate. It is noted that hardware, firmware and/or software elements may be collectively or individually referred to herein as “logic” or “circuit.”

It will be appreciated that the exemplary devices shown in the block diagrams described above may represent one functionally descriptive example of many potential implementations. Accordingly, division, omission or inclusion of block functions depicted in the accompanying figures does not infer that the hardware components, circuits, software and/or elements for implementing these functions would be necessarily be divided, omitted, or included in embodiments.

At least one computer-readable storage medium may include instructions that, when executed, cause a system to perform any of the computer-implemented methods described herein.

Some embodiments may be described using the expression “one embodiment” or “an embodiment” along with their derivatives. These terms mean that a particular feature, structure, or characteristic described in connection with the embodiment is included in at least one embodiment. The appearances of the phrase “in one embodiment” in various places in the specification are not necessarily all referring to the same embodiment. Moreover, unless otherwise noted the features described above are recognized to be usable together in any combination. Thus, any features discussed separately may be employed in combination with each other unless it is noted that the features are incompatible with each other.

With general reference to notations and nomenclature used herein, the detailed descriptions herein may be presented in terms of program procedures executed on a computer or network of computers. These procedural descriptions and representations are used by those skilled in the art to most effectively convey the substance of their work to others skilled in the art.

A procedure is here, and generally, conceived to be a self-consistent sequence of operations leading to a desired result. These operations are those requiring physical manipulations of physical quantities. Usually, though not necessarily, these quantities take the form of electrical, magnetic or optical signals capable of being stored, transferred, combined, compared, and otherwise manipulated. It proves convenient at times, principally for reasons of common usage, to refer to these signals as bits, values, elements, symbols, characters, terms, numbers, or the like. It should be noted, however, that all of these and similar terms are to be associated with the appropriate physical quantities and are merely convenient labels applied to those quantities.

Further, the manipulations performed are often referred to in terms, such as adding or comparing, which are commonly associated with mental operations performed by a human operator. No such capability of a human operator is necessary, or desirable in most cases, in any of the operations described herein, which form part of one or more embodiments. Rather, the operations are machine operations. Useful machines for performing operations of various embodiments include general purpose digital computers or similar devices.

Some embodiments may be described using the expression “coupled” and “connected” along with their derivatives. These terms are not necessarily intended as synonyms for each other. For example, some embodiments may be described using the terms “connected” and/or “coupled” to indicate that two or more elements are in direct physical or electrical contact with each other. The term “coupled,” however, may also mean that two or more elements are not in direct contact with each other, but yet still co-operate or interact with each other.

Various embodiments also relate to apparatus or systems for performing these operations. This apparatus may be specially constructed for the required purpose or it may comprise a general purpose computer as selectively activated or reconfigured by a computer program stored in the computer. The procedures presented herein are not inherently related to a particular computer or other apparatus. Various general purpose machines may be used with programs written in accordance with the teachings herein, or it may prove convenient to construct more specialized apparatus to perform the required method steps. The required structure for a variety of these machines will appear from the description given.

It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separate embodiment. In the appended claims, the terms “including” and “in which” are used as the plain-English equivalents of the respective terms “comprising” and “wherein,” respectively. Moreover, the terms “first,” “second,” “third,” and so forth, are used merely as labels, and are not intended to impose numerical requirements on their objects.

What has been described above includes examples of the disclosed architecture. It is, of course, not possible to describe every conceivable combination of components and/or methodologies, but one of ordinary skill in the art may recognize that many further combinations and permutations are possible. Accordingly, the novel architecture is intended to embrace all such alterations, modifications and variations that fall within the spirit and scope of the appended claims.

Although the present invention has been described above and is defined in the enclosed claims, it should be understood that the present invention can alternatively be defined according to the following embodiments:

1. A method comprising: receiving, at a controller for an automated drug delivery device, one or more indications of drug delivery from the automated drug delivery device; calculating, using a processor of the controller, an amount of progress towards a target system performance of the automated drug delivery device based on the one or more indications of drug delivery; and displaying, on a display of the controller, the amount of progress, wherein the amount of progress is divided into an onboarding period, a dynamic, automatic titration period, and a target system performance period.

2. The method of embodiment 1, wherein the onboarding period is a predetermined fixed length of usage.

3. The method of embodiment 1 or 2, wherein the dynamic, automatic titration period is a predetermined fixed length of usage following the onboarding period.

4. The method of embodiment 1 or 2, wherein the dynamic, automatic titration period is calculated based on a rate of change in a parameter being adapted.

5. The method of embodiment 4, wherein the parameter being adapted is a user's total daily insulin use.

6. The method of any one of embodiments 1 to 5, wherein the dynamic, automatic titration period is calculated, at least in part, based on a comparison of a user's current mean glucose value as compared to a target mean glucose value for the user, the target mean glucose value representing an ideal diabetes control scenario.

7. The method of any one of embodiments 1 to 6, wherein the dynamic, automatic titration period is calculated, at least in part, based on a target time-in-range for the user's glucose level over a predetermined period of time.

8. A non-transitory computer-readable storage medium, the computer-readable storage medium including instructions that when executed by a controller for an automated drug delivery device, cause the controller to: receive, at the controller, one or more indications of drug delivery from the automated drug delivery device; calculate, using a processor of the controller, an amount of progress towards a target system performance of the automated drug delivery device based on the one or more indications of drug delivery; and display, on a display of the controller, the amount of progress, wherein the amount of progress is divided into an onboarding period, a dynamic, automatic titration period, and a target system performance period.

9. The computer-readable storage medium of embodiment 8, wherein the onboarding period is a predetermined fixed length of usage.

10. The computer-readable storage medium of embodiment 8 or 9, wherein the dynamic, automatic titration period is a predetermined fixed length of usage follow the onboarding period.

11. The computer-readable storage medium of embodiment 8 or 9, wherein the dynamic, automatic titration period is calculated based on a rate of change in a parameter being adapted.

12. The computer-readable storage medium of embodiment 11, wherein the parameter being adapted is a user's total daily insulin use.

13. The computer-readable storage medium of any one of embodiments 8 to 12, wherein the dynamic, automatic titration period is calculated, at least in part, based on a comparison of a user's current mean glucose value as compared to a target mean glucose value for the user, the target mean glucose value represents an ideal diabetes control scenario.

14. The computer-readable storage medium of any one of embodiments 8 to 13, wherein the dynamic, automatic titration period is calculated, at least in part, based on a target time-in-range for the user's glucose level over a predetermined period of time.

15. A system comprising: an automated drug delivery device comprising: a reservoir storing a liquid drug, a delivery system for delivering the liquid drug to a user, a processor for calculating dosage amounts for the liquid drug, and a transmitter/receiver configured to transmit the dosage amounts; and a controller comprising: a transmitter/receiver, a display, a processor, and a memory storing instructions that, when executed by the processor, configure the controller to: receive, at the transmitter/receiver of the controller, the dosage amounts from the automated drug delivery device; calculate an amount of progress towards a target system performance of the automated drug delivery device based on the dosage amounts; and display, on the display of the controller, the amount of progress, wherein the amount of progress is divided into an onboarding period, a dynamic, automatic titration period, and a target system performance period.

16. The system of embodiment 15, wherein the onboarding period is a predetermined fixed length of usage.

17. The computing apparatus of embodiment 15 or 16, wherein the dynamic, automatic titration period is: a predetermined fixed length of usage follow the onboarding period, or calculated based on a rate of change in a parameter being adapted

18. The computing apparatus of embodiment 17, wherein the parameter being adapted is a user's total daily insulin use.

19. The computing apparatus of any one of embodiments 15 to 18, wherein the dynamic, automatic titration period is calculated, at least in part, based on a comparison of a user's current mean glucose value as compared to a target mean glucose value for the user, the target mean glucose value represent an ideal diabetes control scenario.

20. The computing apparatus of any one of embodiments 15 to 19, wherein the dynamic, automatic titration period is calculated, at least in part, based on a target time-in-range for the user's glucose level over a predetermined period of time.

21. A method comprising: receiving, at a controller for an automated drug delivery device, one or more blood glucose readings from a sensor of the automated drug delivery device; determining, based on the one or more blood glucose readings, that a sub-optimal glucose control condition exists; and displaying, on a display of the controller, a recommendation based on the sub-optimal glucose control condition.

22. The method of embodiment 21, wherein determining that a sub-optimal glucose control condition exists comprises one or more of: determining that an amount of time that a user has spent in a hyperglycemic range exceeds a predetermined hyperglycemic threshold amount; or determining that an amount of time that a user has spent in a hypoglycemic range exceeds a predetermined hypoglycemic threshold amount.

23. The method of embodiment 21 or 22, wherein determining that a sub-optimal glucose control condition exists comprises one or more of: determining that the user has experienced more than a predetermined number of instances of glucose concentrations above a predetermined upper threshold amount; or determining that the user has experienced more than a predetermined number of instances of glucose concentrations below a predetermined lower threshold amount.

24. The method of any one of embodiments 21 to 23, wherein determining that a sub-optimal glucose control condition exists comprises determining that the user has experienced an average glucose concentration that is higher than a predetermined average concentration threshold amount.

25. The method of any one of embodiments 21 to 24, wherein the recommendation comprises an expected best-case blood glucose control scenario.

26. The method of any one of embodiments 21 to 25, wherein the recommendation comprises a suggested change to the user's basal dose.

27. The method of any one of embodiments 21 to 26, wherein the recommendation comprises a suggested change to the user's bolus dose.

28. The method of any one of embodiments 21 to 27, further comprising displaying a relationship between the user's average bolus per day and/or bolus ratio to the user's time-in-range.

29. A non-transitory computer-readable storage medium, the computer-readable storage medium including instructions that when executed by a controller for an automated drug delivery device, cause the computer to: receive, at the controller, one or more blood glucose readings from a sensor of the automated drug delivery device; determine, based on the one or more blood glucose readings, that a sub-optimal glucose control condition exists; and display, on a display of the controller, a recommendation based on the sub-optimal glucose control condition.

30. The computer-readable storage medium of embodiment 29, wherein determining that a sub-optimal glucose control condition exists comprises one or more of: determine that an amount of time that a user has spent in a hyperglycemic range exceeds a predetermined hyperglycemic threshold amount; or determine that an amount of time that a user has spent in a hypoglycemic range exceeds a predetermined hypoglycemic threshold amount.

31. The computer-readable storage medium of embodiment 29 or 30, wherein determining that a sub-optimal glucose control condition exists comprises one or more of: determine that the user has experienced more than a predetermined number of instances of glucose concentrations above a predetermined upper threshold amount; or determine that the user has experienced more than a predetermined number of instances of glucose concentrations below a predetermined lower threshold amount.

32. The computer-readable storage medium of any one of embodiments 29 to 31, wherein determine that a sub-optimal glucose control condition exists comprises determining that the user has experienced an average glucose concentration that is higher than a predetermined average concentration threshold amount.

33. The computer-readable storage medium of any one of embodiments 29 to 32, wherein the recommendation comprises an expected best-case blood glucose control scenario.

34. The computer-readable storage medium of any one of embodiments 29 to 33, wherein the recommendation comprises a suggested change to the user's basal dose.

35. The computer-readable storage medium of any one of embodiments 29 to 34, wherein the recommendation comprises a suggested change to the user's bolus dose.

36. The computer-readable storage medium of any one of embodiments 29 to 35, wherein the instructions further configure the computer to display a relationship between the user's average bolus per day and/or bolus ratio to the user's time-in-range.

37. A system comprising: an automated drug delivery device comprising: a sensor configured to detect a blood glucose level of a user; a processor for calculating the user's blood glucose level, and a transmitter/receiver configured to transmit the dosage amounts; and a controller comprising: a transmitter/receiver, a display, a processor, and a memory storing instructions that, when executed by the processor, configure the controller to: receive one or more blood glucose readings from a sensor of the automated drug delivery device; determine, based on the one or more blood glucose readings, that a sub-optimal glucose control condition exists; and display, on a display of the controller, a recommendation based on the sub-optimal glucose control condition.

38. The system of embodiment 37, wherein determining that a sub-optimal glucose control condition exists comprises one or more of: determining that an amount of time that a user has spent in a hyperglycemic range exceeds a predetermined hyperglycemic threshold amount; determining that an amount of time that a user has spent in a hypoglycemic range exceeds a predetermined hypoglycemic threshold amount; determining that the user has experienced more than a predetermined number of instances of glucose concentrations above a predetermined upper threshold amount; determining that the user has experienced more than a predetermined number of instances of glucose concentrations below a predetermined lower threshold amount; or determining that a sub-optimal glucose control condition exists comprises determining that the user has experienced an average glucose concentration that is higher than a predetermined average concentration threshold amount.

39. The system of embodiment 37 or 38, wherein the recommendation comprises: an expected best-case blood glucose control scenario; a suggested change to the user's basal dose; or a suggested change to the user's bolus dose.

40. The system of one of embodiments 37 to 39, wherein the instructions further configure the apparatus to display a relationship between the user's average bolus per day and/or bolus ratio to the user's time-in-range.

What is claimed is:

1. A method comprising:
receiving, at a controller for an automated drug delivery device, one or more indications of drug delivery from the automated drug delivery device;
calculating, using a processor of the controller, an amount of progress towards a target system performance of the automated drug delivery device based on the one or more indications of drug delivery; and
displaying, on a display of the controller, the amount of progress toward target system performance.

2. The method of claim 1, wherein the amount of progress is divided into an onboarding period, a dynamic, automatic titration period, and a target system performance period and the onboarding period is a predetermined fixed length of usage.

3. The method of claim 2, wherein the dynamic, automatic titration period is a predetermined fixed length of usage following the onboarding period.

4. The method of claim 2, wherein the dynamic, automatic titration period is calculated based on a rate of change in a parameter being adapted.

5. The method of claim 4, wherein the parameter being adapted is a user's total daily insulin use.

6. The method of claim 2, wherein the dynamic, automatic titration period is calculated, at least in part, based on a comparison of a user's current mean glucose value as compared to a target mean glucose value for the user, the target mean glucose value representing an ideal diabetes control scenario.

7. The method of claim 2, wherein the dynamic, automatic titration period is calculated, at least in part, based on a target time-in-range for the user's glucose level over a predetermined period of time.

8. A non-transitory computer-readable storage medium, the computer-readable storage medium including instructions that when executed by a controller for an automated drug delivery device, cause the controller to:
receive, at the controller, one or more indications of drug delivery from the automated drug delivery device;
calculate, using a processor of the controller, an amount of progress towards a target system performance of the automated drug delivery device based on the one or more indications of drug delivery; and
display, on a display of the controller, the amount of progress.

9. The computer-readable storage medium of claim 8, wherein the amount of progress is divided into an onboarding period, a dynamic, automatic titration period, and a target system performance period and the onboarding period is a predetermined fixed length of usage.

10. The computer-readable storage medium of claim 9, wherein the dynamic, automatic titration period is a predetermined fixed length of usage follow the onboarding period.

11. The computer-readable storage medium of claim 9, wherein the dynamic, automatic titration period is calculated based on a rate of change in a parameter being adapted.

12. The computer-readable storage medium of claim 11, wherein the parameter being adapted is a user's total daily insulin use.

13. The computer-readable storage medium of claim 9, wherein the dynamic, automatic titration period is calculated, at least in part, based on a comparison of a user's current mean glucose value as compared to a target mean glucose value for the user, the target mean glucose value representing an ideal diabetes control scenario.

14. The computer-readable storage medium of claim 9, wherein the dynamic, automatic titration period is calculated, at least in part, based on a target time-in-range for the user's glucose level over a predetermined period of time.

15. A system comprising:
an automated drug delivery device comprising:
a reservoir storing a liquid drug,
a delivery system for delivering the liquid drug to a user,
a processor for calculating dosage amounts for the liquid drug, and
a transmitter/receiver configured to transmit the dosage amounts; and
a controller comprising:
a transmitter/receiver,
a display,
a processor, and
a memory storing instructions that, when executed by the processor, configure the controller to:
receive, at the transmitter/receiver of the controller, the dosage amounts from the automated drug delivery device;
calculate an amount of progress towards a target system performance of the automated drug delivery device based on the dosage amounts; and
display, on the display of the controller, the amount of progress.

16. The system of claim 15, wherein the amount of progress is divided into an onboarding period, a dynamic, automatic titration period, and a target system performance period and the onboarding period is a predetermined fixed length of usage.

17. The system of claim 16, wherein the dynamic, automatic titration period is:
a predetermined fixed length of usage follow the onboarding period, or
calculated based on a rate of change in a parameter being adapted.

18. The system of claim 17, wherein the parameter being adapted is a user's total daily insulin use.

19. The system of claim 16, wherein the dynamic, automatic titration period is calculated, at least in part, based on a comparison of a user's current mean glucose value as compared to a target mean glucose value for the user, the target mean glucose value represent an ideal diabetes control scenario.

20. The system of claim 16, wherein the dynamic, automatic titration period is calculated, at least in part, based on a target time-in-range for the user's glucose level over a predetermined period of time.
